(19) United States
(12) Patent Application Publication
(10) Pub. No.: US 2024/0327882 A1
(43) Pub. Date: Oct. 3, 2024

(54) Methods and Compositions for Increasing Bio-products in Cyanobacteria using Low-Dose Antibiotics

(71) Applicant: Morgan State University, Baltimore, MD (US)

(72) Inventors: Viji Sitther, Baltimore, MD (US); Yavuz Yalcin, Baltimore, MD (US); Busra Aydin, Baltimore, MD (US)

(21) Appl. No.: 18/592,932

(22) Filed: Mar. 1, 2024

Related U.S. Application Data
(63) Continuation-in-part of application No. 18/430,193, filed on Feb. 1, 2024.
(60) Provisional application No. 63/449,076, filed on Mar. 1, 2023.

(51) Int. Cl. C12P 7/6409; C12N 1/20; C12R 1/01

(52) U.S. Cl. CPC C12P 7/6409; C12N 1/205; C12R 2001/01

(57) ABSTRACT

Methods and compositions are detailed for increasing bio-products in cyanobacteria where the cyanobacteria is incubated in the presence of non-lethal amounts of antibiotic. Non-lethal doses of antibiotic weaken the cell membrane facilitating migration of fatty acids from the cell, reducing inhibition feedback, leading to an increase in bio-products.

### GOVERNMENT RIGHTS

This invention was made with government support under grant number NSF-EiR 1900966, awarded by the National Science Foundation. The government has certain rights in the invention.

### FIELD OF THE INVENTION

This invention relates to methods and compositions for increasing bio-products in cyanobacteria using low-dose antibiotics.

### BACKGROUND OF THE INVENTION

In recent years, increased antibiotic contamination in surface and groundwater has drawn worldwide attention due to their potential consequences for the environmental ecosystem and health. Globally, antibiotic consumption has increased by 64% and at the rate of 39% over the past two decades. In the United States alone, about 10,000 tons per annum of antibiotics are consumed and account for ˜70% of the nation's annual antimicrobial consumption. Antibiotic residues excreted in urine and feces after metabolism are directly introduced to the aquatic environments by poorly managed livestock that have direct access to surface water or indirectly by animal manure. While non-targeted antibiotic exposure on eukaryotes is minimal compared to prokaryotes, cyanobacteria are ten times more sensitive than algae to the harmful effects of antibiotics because of fragile cell structures. About 20 different kinds of antibiotics have been detected in the range of 1.26-127.49 mg/L in various aquatic environments. Of these, the α-lactam group, primarily the penicillin and cephalosporins, constitutes 50-60% of the most consumed antibiotics. Significant amounts of β-lactams are directly excreted without any structural changes after metabolism. Besides, antibiotic residues are detrimental to microbial communities in aquatic ecosystems and are known to greatly impact cellular metabolism in cyanobacteria. The hormesis phenomenon (biphasic effect) in response to harmful environmental agents by low-dose stimulation and high-dose inhibition has been extensively studied. Exposure of Microcystis aeruginosa to low dosages (<20 mg/L) of erythromycin has been reported to trigger photosynthetic activity.

Of the various cyanobacterial strains, Fremyella diplosiphon is a widely studied model organism known for its adaptive growth capability in varying light intensities. Besides, its ability to produce lipids and desirable essential fatty acids make it an ideal third generation biofuel agent. In addition, numerous useful bio-products and/or metabolites of cyanobacteria have beneficial bioactivities, including alkaloids, depsipeptides, lipopeptides, macrolides/lactones, peptides, terpenes, polysaccharides, lipids, polyketides, and others. Heretofore, the impact of antibiotics on F. diplosiphon growth and cell membrane permeability remains unknown.

### SUMMARY OF THE INVENTION

The impact of ampicillin, tetracycline, kanamycin, and cefotaxime antibiotic exposure on structural and metabolic changes, cell membrane permeability, growth and pigment accumulation in F. diplosiphon strains B481-SD (engineered to produce lipids) and B481-WT (wild type) was investigated, together with their impact on fatty acid and other bio-product production. Enhanced membrane permeability detected by lactate dehydrogenase assay indicated maximal activity at 0.8 mg/L ampicillin, kanamycin, and tetracycline treatments on day 6. Both strains exposed to kanamycin from 0.2 to 3.2 mg/L and tetracycline from 0.8 to 12.8 mg/L enhanced pigment accumulation. Additionally, B-481-SD treated with ampicillin from 0.2 to 51.2 mg/L exhibited a significant enhancement (p<0.005) in pigment fluorescence. A detrimental effect on pigmentation was observed in both strains treated with kanamycin from 6.4 to 102.5 mg/L and cefotaxime from 0.8 to 102.5 mg/L. Morphological alterations in cells exposed to antibiotics revealed abundant vacuolation, pyrophosphate, and cyanophycin granule formation in treated cells as a response to antibiotic stress. FAME components, including hexadecanoic acid (C16: 0), methyloctadecenoate (C18: 1) methyl octadecadienoate (C18: 2) and ç-Linolenic acid (C18: 3), were observed to have significantly increased compared to the untreated group. Enhanced amounts of saturated fatty acids, polyunsaturated fatty acids, and monounsaturated fatty acids were detected in 0.8 mg/L ampicillin treated cultures. These findings on the hormetic effect of antibiotics on F. diplosiphon indicate that optimal antibiotic concentrations induce cellular growth and pigment accumulation, and bio-product production, while high concentrations severely impact cellular functionality. Growth was significantly inhibited at concentrations above 51.2 mg/L.

Therefore, according to various embodiments of the invention, methods for increasing pigmentation and bio-product production of cyanobacteria comprising growing cultures of said cyanobacteria in the presence of non-lethal levels of antibiotics is provided. The cyanobacteria may be F. diplosiphon, exemplary strains of which include strains B481-SD and B481-WT. Antibiotics according to the invention may include β-lactams, aminoglycosides or tetracyclines. Specific antibiotics may include ampicillin, tetracycline, or kanamycin. Optimal antibiotic concentration may be determined by lactate dehydrogenase activity. Cyanobacteria bio-product production which may be increased by the methods of the invention include increased production of alkaloids, depsipeptides, lipopeptides, macrolides/lactones, peptides, terpenes, polysaccharides, lipids, polyketides, among others. Lipid production which is increased by the methods of the invention may include one or more of saturated fatty acids, polyunsaturated fatty acids, and monounsaturated fatty acids.

There is further provided according to the invention a composition comprising cyanobacteria comprising a cyanobacteria and a non-lethal amount of an antibiotic. The cyanobacteria may be F. diplosiphon, including strains B481-SD or B481-WT. Antibiotics according to the invention may include β-lactams, aminoglycosides or tetracyclines. Specific antibiotics may include ampicillin, tetracycline, or kanamycin. The antibiotic may be present in the composition in concentrations of about 0.2 mg/L to about 0.8 mg/L, about 0.4 mg/L to about 0.8 mg/L and most preferably about 0.8 mg/L. Compositions according to the invention may further comprise fatty acid methyl ester in greater amounts as compared to cyanobacteria compositions grown in the absence of antibiotic. Such fatty acid methyl esters may include saturated fatty acids, polyunsaturated fatty acids, and monounsaturated fatty acids in increased amounts compared to cyanobacteria compositions grown in the absence of antibiotic. In particular, compositions of the present invention may comprise increased amounts of hexadecanoic acid (C16: 0), methyloctadecenoate (C18: 1) methyl octadecadienoate (C18: 2) and ç-Linolenic acid (C18: 3), as compared to cyanobacteria compositions grown in the absence of antibiotic.

### BRIEF DESCRIPTION OF DRAWINGS

FIG. 1A shows lactate dehydrogenase activity in B481-SD strain of F. diplosiphon exposed to varying concentrations of ampicillin, kanamycin, and tetracycline on day 6. Mean and standard deviations are indicated by error bars.

FIG. 1B shows lactate dehydrogenase activity in B481-WT strain of F. diplosiphon exposed to varying concentrations of ampicillin, kanamycin, and tetracycline on day 6. Mean and standard deviations are indicated by error bars.

FIG. 2 shows morphological alterations of F. diplosiphon strains in concentrations at 25.6 mg/L ampicillin (frames A and B), 51.2 mg/L kanamycin (frames C and D) and 102.5 mg/L tetracycline (frames E and F) treatments. Representative sections of color bright field images were captured using a Cytation 5 Cell Imaging Multi-Mode reader at 40× magnification bars, 100 μm.

FIG. 3 is a chart showing a comparison of fatty acid methyl ester (FAME) components including hexadecanoic acid (C16: 0), methyloctadecenoate (C18: 1) methyl octadecadienoate (C18: 2) and ç-Linolenic acid (C18: 3) abundance in F. displosiphon treated with 0.8 mg/L ampicillin and untreated control.

FIG. 4 is a chart showing saturated and unsaturated fatty acid methyl ester (FAME) percentages in the total FAMEs of F. displosiphon untreated control and cultures treated with 0.8 mg/L ampicillin. *SFAs (saturated fatty acids), PUFAs (polyunsaturated fatty acids), MUFAS (monounsaturated fatty acids).

FIG. 5 is a chart showing phycocyanin autofluorescence of B481-SD reflected by 590 nm excitation and 650 nm emission following exposure to ampicillin at concentrations ranging from 0.2 to 102.5 mg/L.

FIG. 6 is a chart showing phycocyanin autofluorescence of B481-SD reflected by 590 nm excitation and 650 nm emission following exposure to cefotaxime at concentrations ranging from 0.2 to 102.5 mg/L.

FIG. 7 is a chart showing phycocyanin autofluorescence of B481-SD reflected by 590 nm excitation and 650 nm emission following exposure to kanamycin at concentrations ranging from 0.2 to 102.5 mg/L.

FIG. 8 is a chart showing phycocyanin autofluorescence of B481-SD reflected by 590 nm excitation and 650 nm emission following tetracycline to ampicillin at concentrations ranging from 0.2 to 102.5 mg/L.

FIG. 9 is a chart showing phycocyanin autofluorescence of B481-WT following exposure to ampicillin at concentrations ranging from 0.2 to 102.5 mg/L.

FIG. 10 is a chart showing phycocyanin autofluorescence of B481-WT following exposure to cefotaxime at concentrations ranging from 0.2 to 102.5 mg/L.

FIG. 11 is a chart showing phycocyanin autofluorescence of B481-WT following exposure to kanamycin at concentrations ranging from 0.05 to 102.5 mg/L.

FIG. 12 is a chart showing phycocyanin autofluorescence of B481-WT following exposure to tetracycline at concentrations ranging from 0.2 to 410 mg/L.

FIG. 13 is a chart showing chlorophyll α autofluorescence of F. diplosiphon strain B481-SD reflected by 420 nm excitation and 680 nm emission following exposure to ampicillin at concentrations ranging from 0.2 to 102.5 mg/L.

FIG. 14 is a chart showing chlorophyll α autofluorescence of F. diplosiphon strain B481-SD reflected by 420 nm excitation and 680 nm emission following exposure to cefotaxime at concentrations ranging from 0.2 to 102.5 mg/L

FIG. 15 is a chart showing chlorophyll α autofluorescence of F. diplosiphon strain B481-SD reflected by 420 nm excitation and 680 nm emission following exposure to kanamycin at concentrations ranging from 0.2 to 102.5 mg/L.

FIG. 16 is a chart showing chlorophyll α autofluorescence of F. diplosiphon strain B481-SD reflected by 420 nm excitation and 680 nm emission following exposure to tetracycline at concentrations ranging from 0.2 to 102.5 mg/L.

FIG. 17 is a chart showing chlorophyll α autofluorescence of F. diplosiphon strain B481-WT reflected by 420 nm excitation and 680 nm emission following exposure to ampicillin at concentrations ranging from 0.2 to 102.5 mg/L.

FIG. 18 is a chart showing chlorophyll α autofluorescence of F. diplosiphon strain B481-WT reflected by 420 nm excitation and 680 nm emission following exposure to cefotaxime at concentrations ranging from 0.2 to 102.5 mg/L.

FIG. 19 is a chart showing chlorophyll α autofluorescence of F. diplosiphon strain B481-WT reflected by 420 nm excitation and 680 nm emission following exposure to kanamycin at concentrations ranging from 0.05 to 102.5 mg/L.

FIG. 20 is a chart showing chlorophyll α autofluorescence of F. diplosiphon strain B481-WT reflected by 420 nm excitation and 680 nm emission following exposure to tetracycline at concentrations ranging from 0.2 to 410 mg/L.

FIG. 21 is a chart showing variance of Fv/Fo in F. diplosiphon strain B481-SD in response to ampicillin exposure for 6 days. Mean and standard deviations are indicated by error bars.

FIG. 22 is a chart showing variance of Fv/Fo in F. diplosiphon strain B481-WT in response to ampicillin exposure for 6 days. Mean and standard deviations are indicated by error bars.

FIG. 23 is a chart showing variance of Fv/Fo in F. diplosiphon strain B481-SD in response to cefotaxime exposure for 6 days. Mean and standard deviations are indicated by error bars.

FIG. 24 is a chart showing variance of Fv/Fo in F. diplosiphon strain B481-WT in response to cefotaxime exposure for 6 days. Mean and standard deviations are indicated by error bars.

FIG. 25 is a chart showing variance of Fv/Fo in F. diplosiphon strain B481-SD in response to kanamycin exposure for 6 days. Mean and standard deviations are indicated by error bars.

FIG. 26 is a chart showing variance of Fv/Fo in F. diplosiphon strain B481-WT in response to kanamycin exposure for 6 days. Mean and standard deviations are indicated by error bars.

FIG. 27 is a chart showing variance of Fv/Fo in F. diplosiphon strain B481-SD in response to tetracycline exposure for 6 days. Mean and standard deviations are indicated by error bars.

FIG. 28 is a chart showing variance of Fv/Fo in F. diplosiphon strain B481-WT in response to tetracycline exposure for 6 days. Mean and standard deviations are indicated by error bars.

### DETAILED DESCRIPTION OF THE INVENTION

The toxicity of ampicillin, cefoxime, tetracycline, kanamycin and zero-valent nanoscale nanoparticles (nZVIs) on F. diplosiphon was first assessed using the Pierce™ LDH Cytotoxicity Assay Kit (Thermo Fisher Scientific, United States) according to the manufacturer's protocol. The assay quantitatively measures released extracellular LDH activity in culture media by enzymatic reaction. Once it was established that F. diplosiphon can tolerate mild levels of antibiotics, the effect of antibiotics on cell membrane permeability, total lipids, and fatty acid methyl esters was demonstrated.

F. diplosiphon strains, B481-WT obtained from the UTEX algae repository (Austin, TX, United States), and B481-SD (overexpressed strain with the sterol desaturase gene; accession MH329183) were used in this study. Cultures were grown in liquid BG-11/HEPES medium under wide-spectrum red light (650 nm) with continuous shaking at 170 rpm at 28° C. in an Innova 44R shaker (Eppendorf, Hamburg, Germany) for 6 days. Light fluorescence rate was adjusted to 30 μmol m⁻² s⁻¹ using the model LI-190SA quantum sensor (Li-Cor, United States). These conditions were kept constant during the study.

Three classes of antibiotics: β-lactams (ampicillin), aminoglycosides (kanamycin), and tetracycline, were tested in this study. Antibiotic stock solutions (25×-100×) were prepared according to the manufacturer's instructions and stored at −20° C. Working solutions in the range of 0.2-102.5 mg/L for ampicillin and kanamycin, 0.05-102.5 mg/L for cefotaxime, 0.2-410 mg/L for tetracycline, and 0.4 to 12.8 mg/L nZVIs were used in this study (Dias et al., 2015; Shang et al., 2015). Ampicillin, cefotaxime, and kanamycin working solutions were prepared immediately before use and diluted in ddH₂O to the desired concentrations (Baselga-Cervera et al., 2019). Each antibiotic concentration was mixed with 5 ml F. diplosiphon cells adjusted to OD750 nm and 200 μl of each dilution added.

Nanofer 25s nZVIs coated with polyacrylic acid were obtained from a nano iron company (Rajhrad, Czech Republic) and adjusted to final concentrations of 6.4 and 12.8 mg/L nZVIs.

Cells grown in the absence of antibiotics or nZVIs served as control. In order to minimize the effects of light scattering, every other well was left blank. Plates were sealed with a Breathe-Easy sealing membrane (Sigma-Aldrich, MO, Lot #MKCP8263) to prevent evaporative water loss and decrease the risk of contamination.

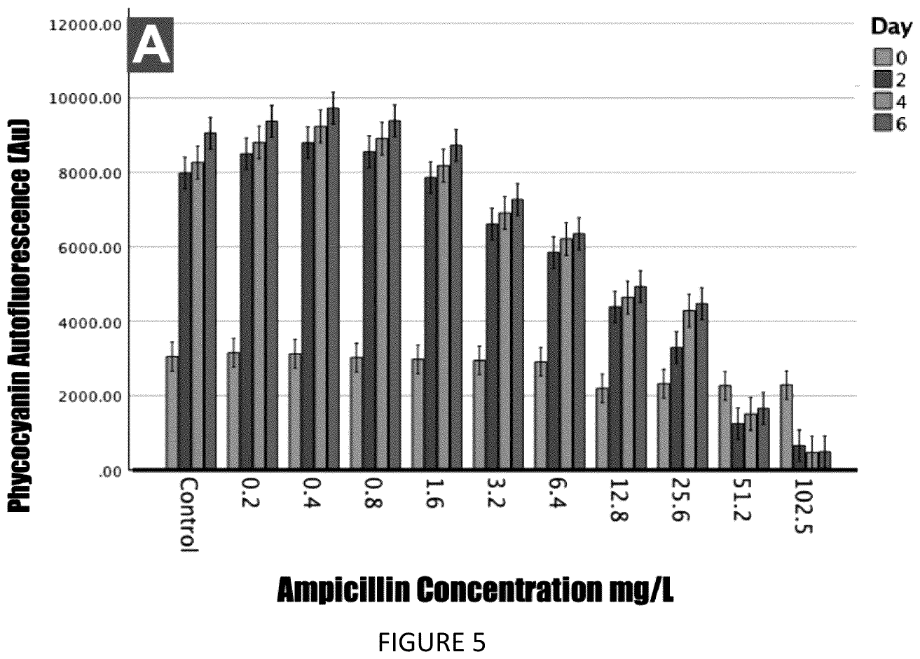

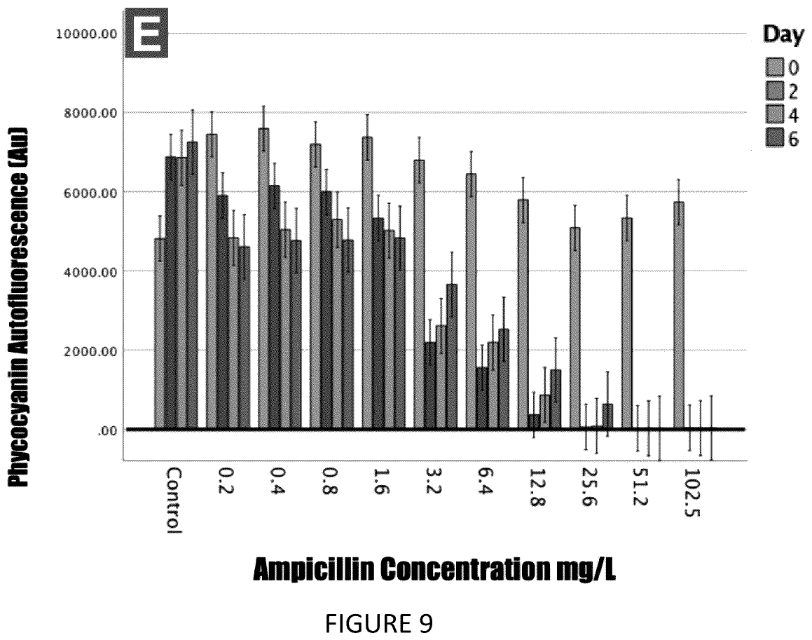

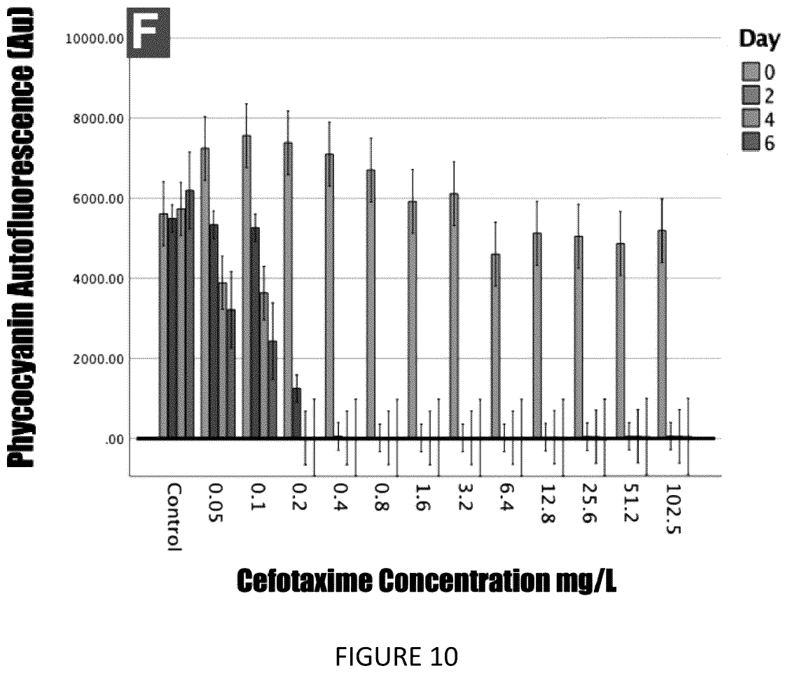

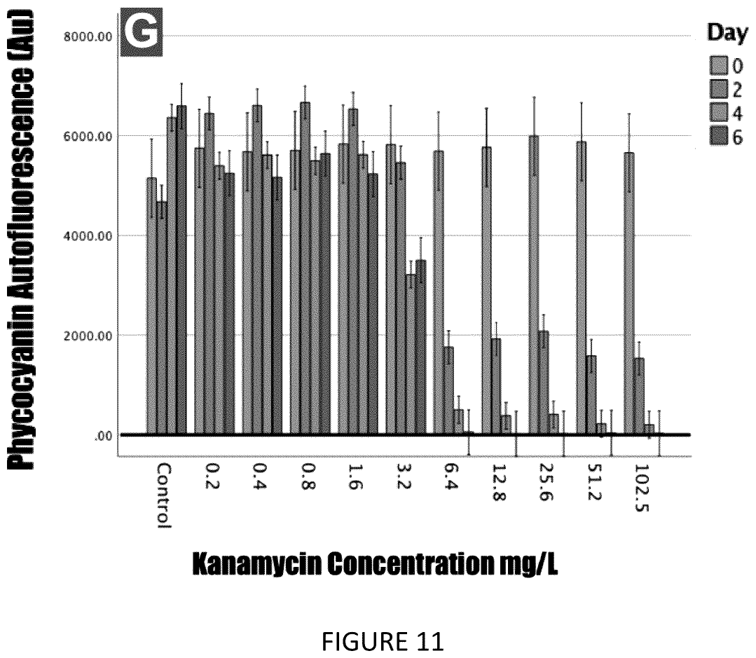

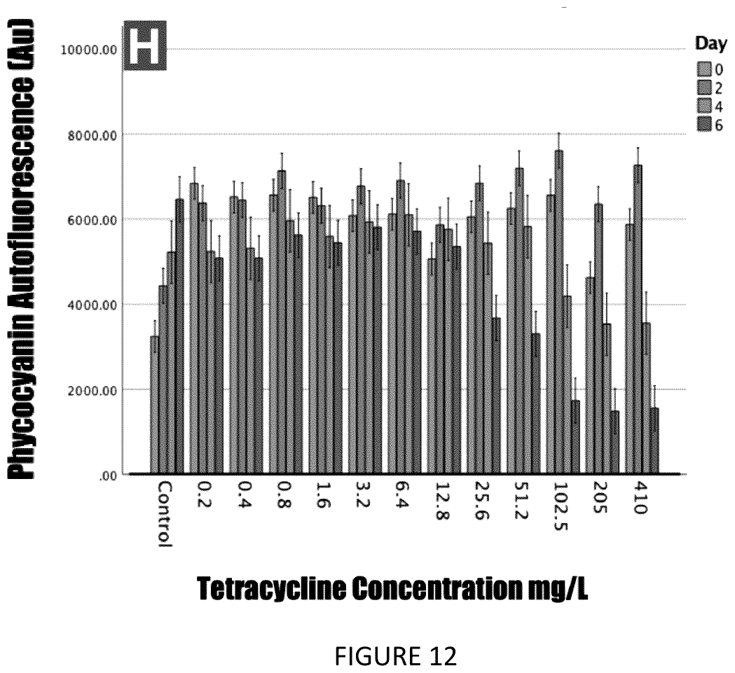

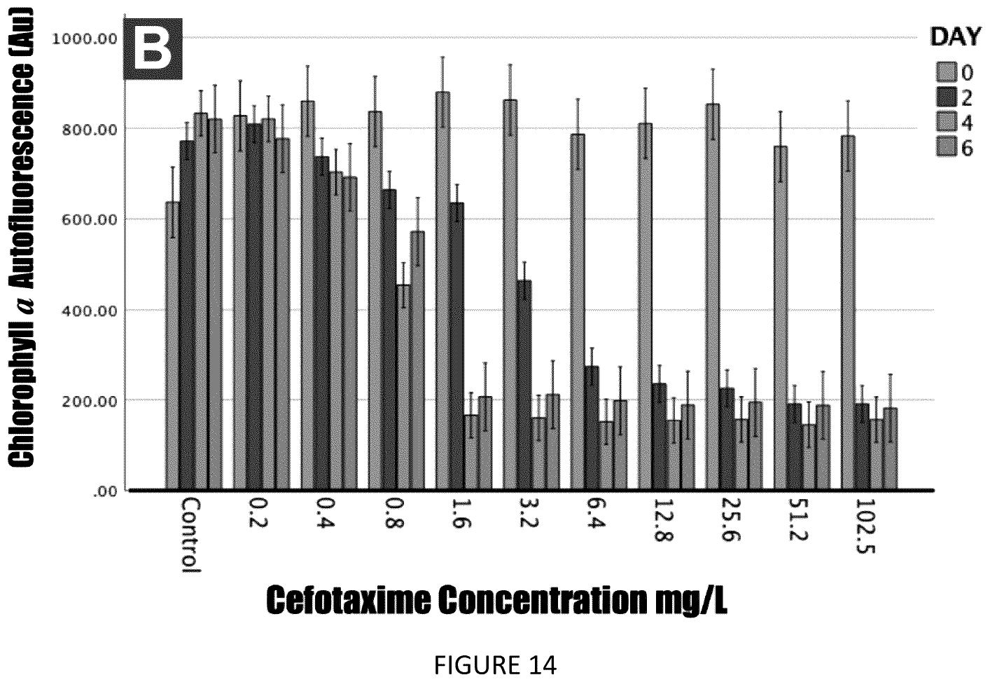

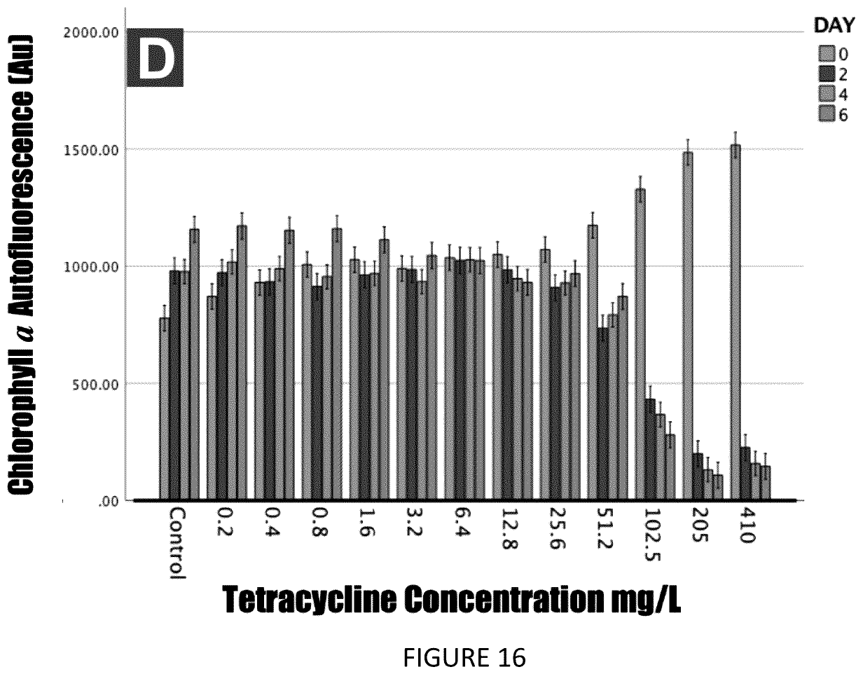

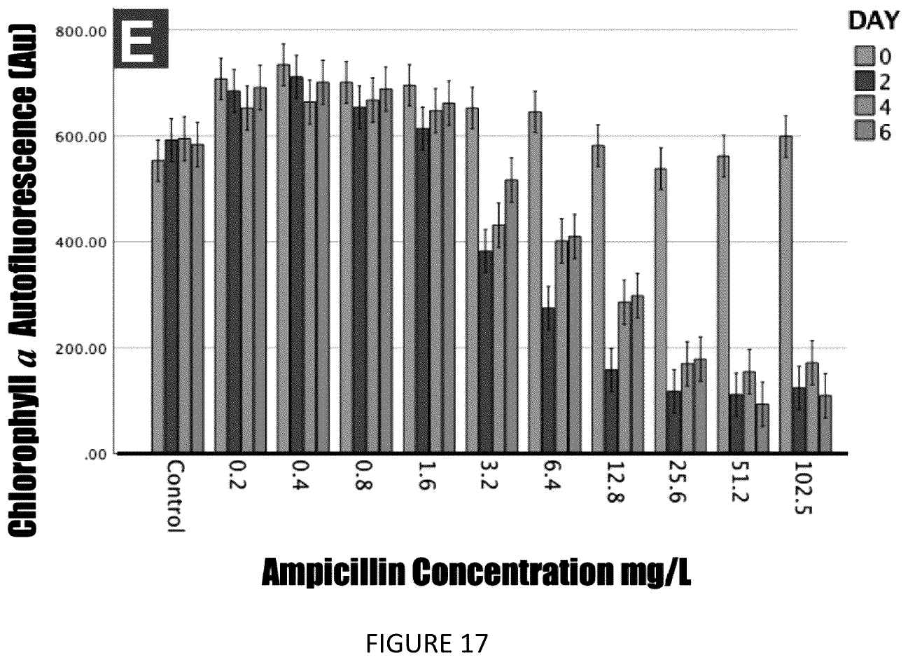

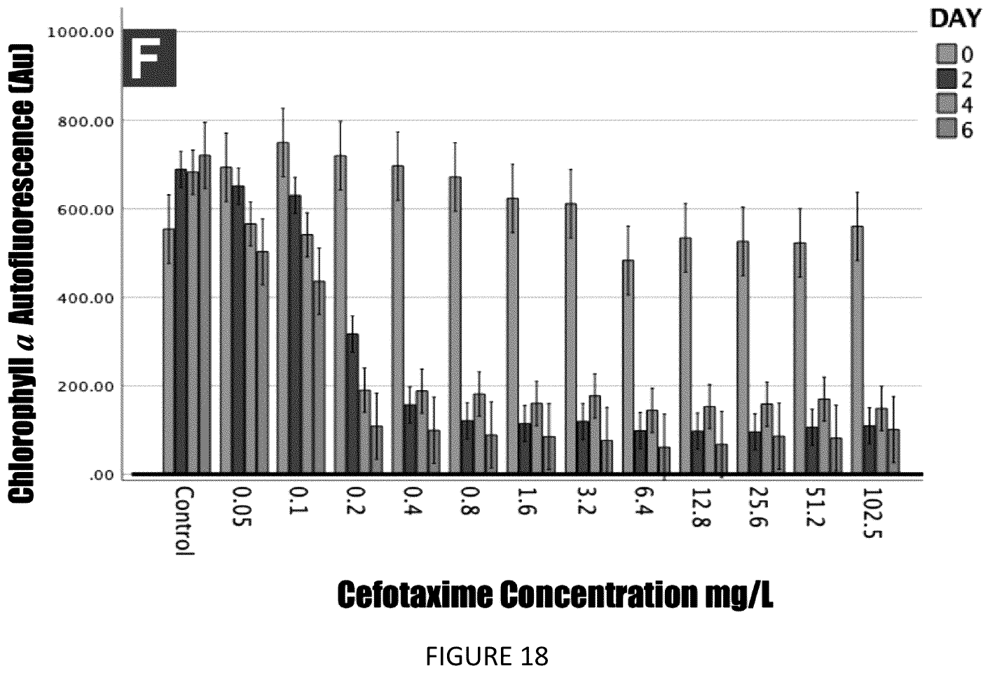

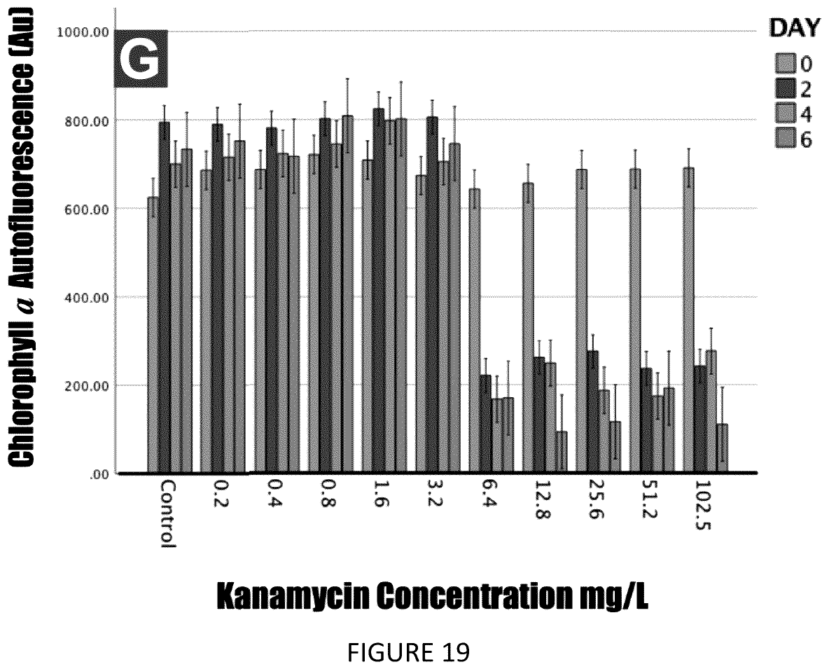

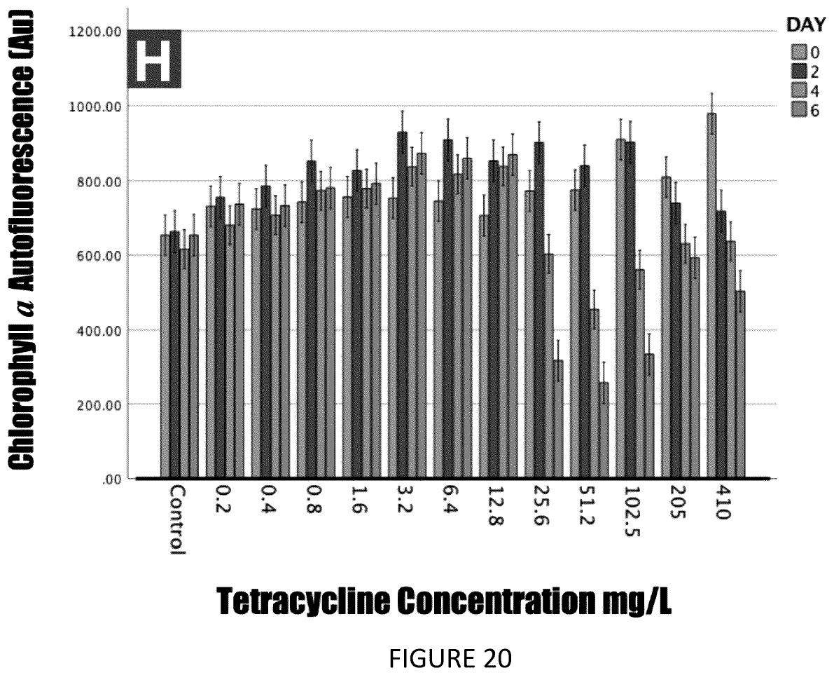

Phycocyanin and chlorophyll α fluorescence in antibiotic-treated and control F. diplosiphon B481-WT and B481-SD were recorded every other day (48 hour intervals) using a microplate reader (BioTek Synergy H1 Microplate Reader, Agilent, USA). While chlorophyll α fluorescence was recorded at an excitation of 420 nm and emission of 680 nm, phycocyanin was measured at excitations of 590 nm, and emissions of 650 nm, see FIGS. 5-20. Maximum PSII quantum yield (Fv/Fo) of treated and controlled F. diplosiphon strains was likewise measured using MINI-PAM (Walz, Effeltrich, Germany) every 48 h for 6 days after incubating in dark for 15 min. The (Fv/Fo) was calculated using the equation

$$\frac{FV}{Fo} = \frac{Fm - Fo}{Fo}$$

The flasks were placed in an incubator at 37° C. in the dark for 24 h prior to lactate dehydrogenase (“LDH”) measurement. On day 6, 50 μl cultures were transferred to a 96 well clear polystyrene microplate (Corning, Inc., NY), and fluorescence Epi-RGB mode was used to macro-evaluation of antibiotic-treated both strains (Amersham Imager 680, GE Healthcare Bio-Sciences AB, Uppsala, Sweden). Next, 50 μL of the reaction mixture (LDH Cytotoxicity Assay Kit, Thermo Fisher Scientific, United States) was added. After incubation for 30 min. at room temperature in the dark, 50 μL of stock solution was added and mixed gently (Wejnerowski et al., 2018). Absorbance was measured at 490 nm using a microplate reader (BioTek Synergy H1 Microplate Reader, Agilent, United States) after 2 h incubation in the dark at room temperature. The calculated average of the control included cells without antibiotics or nVZI treatment was accepted as healthy cells and results were determined by subtracting LDH activity of the control from treated cells. Microscopic observations of antibiotic-related membrane and cyanobacterial structure alterations were made using a Cytation 5 Cell Imaging Multi-Mode reader (BioTek® Instruments, Inc., Winooski, United States).

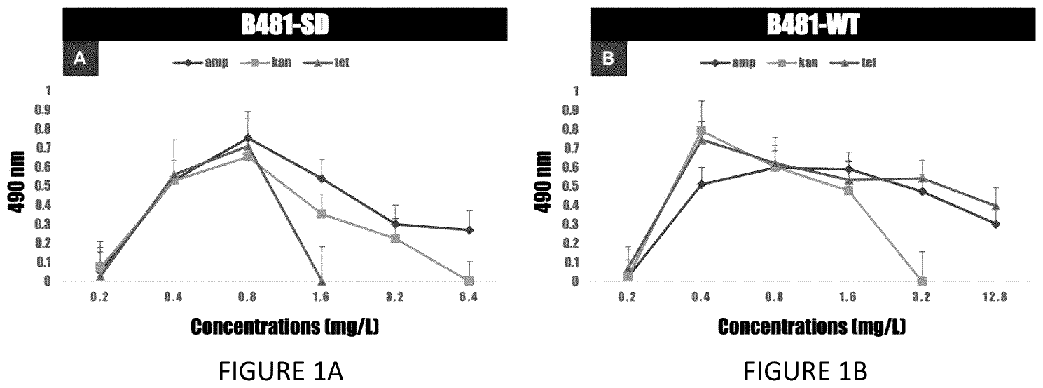

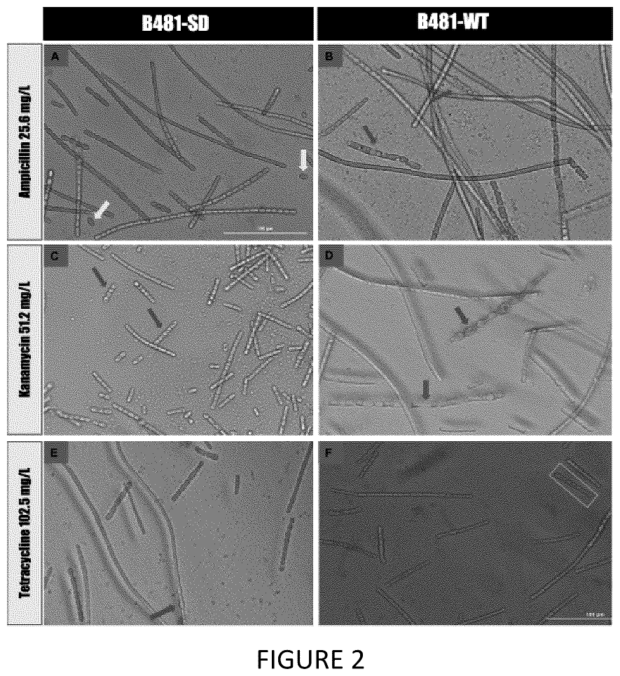

B481-SD and B481-WT strains treated with ampicillin, tetracycline, and kanamycin exhibited maximum LDH activity at the concentrations of 0.8 and 0.4 mg/L, respectively. Specifically, enhanced LDH activity (p<0.05) was observed in B481-SD treated with tetracycline, ampicillin, and kanamycin from 0.2 to 0.8 mg/L (FIG. 1A). The LDH activity of B481-WT was higher in kanamycin and tetracycline at 0.4 mg/L compared to ampicillin at the same concentration (FIG. 1B). Microscopic observations such as filament fragmentation and alteration of cell shape were observed at concentrations higher than 25.6 mg/L ampicillin for B481-WT and 51.2 mg/L kanamycin for B481-SD and B481-WT (FIG. 2, frames B-D). In addition, cellular stress-related structures such as pyrophosphate granules (FIG. 2, frame F, green rectangle), akinetes (FIG. 2, frame A, yellow arrows), and cellular vacuoles (FIG. 2, frames B-F, red arrows) were observed in the strains exposed to higher antibiotic concentrations.

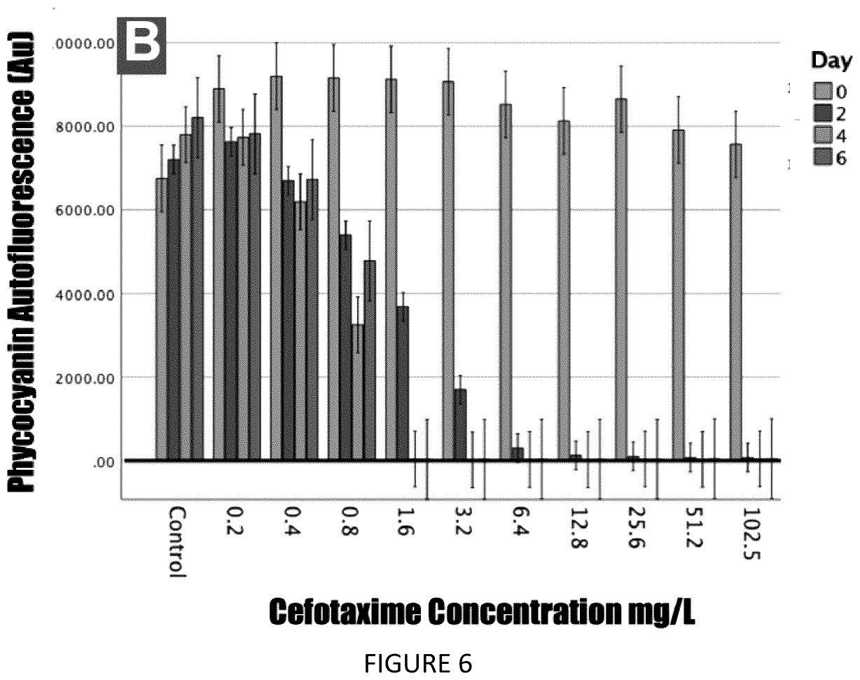

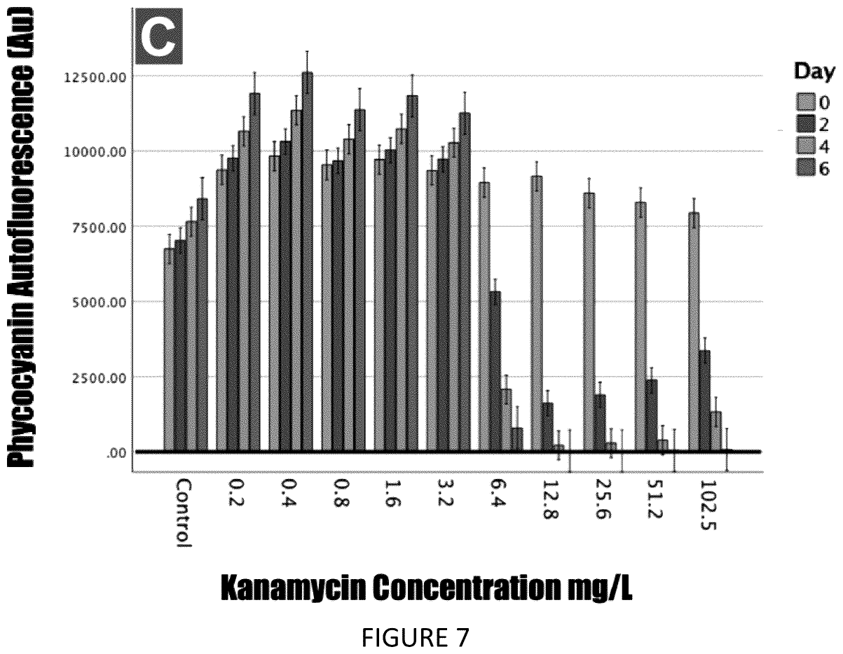

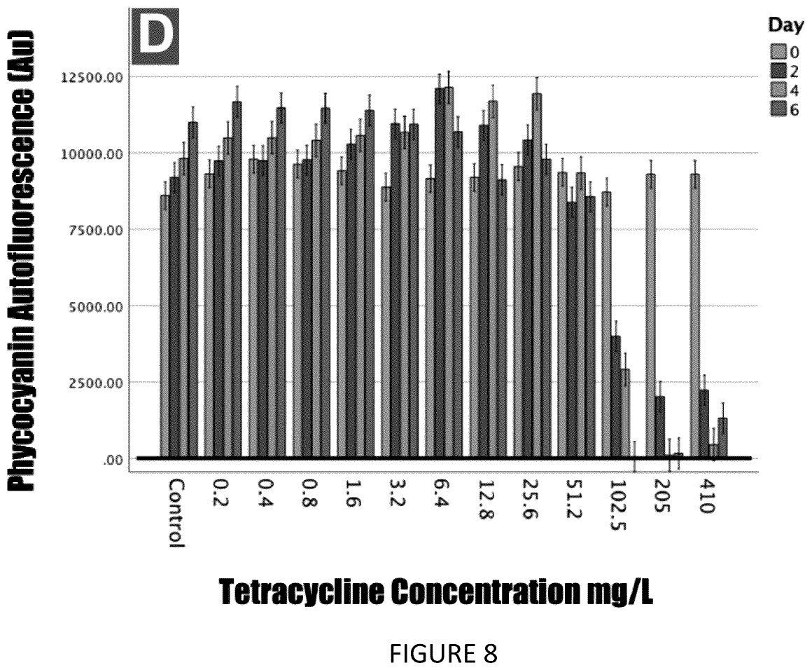

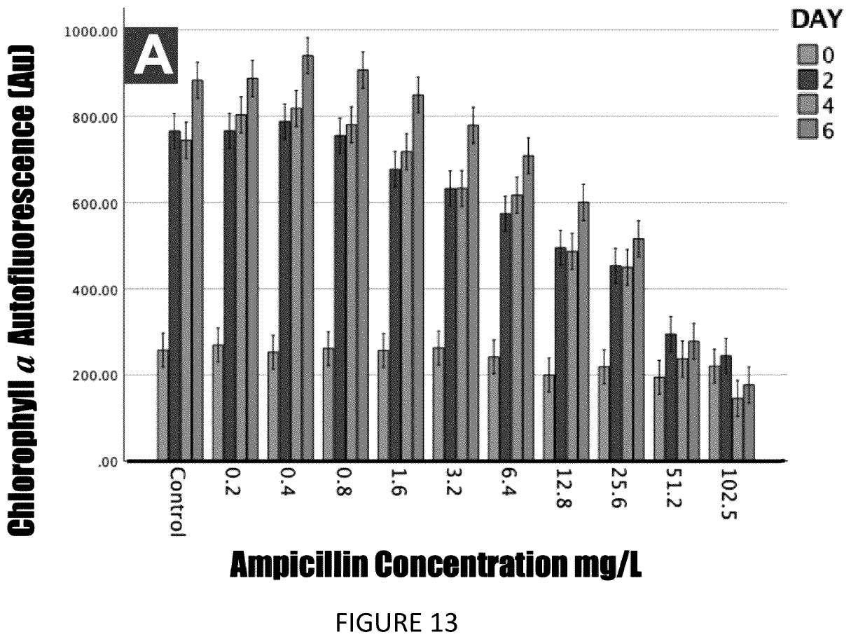

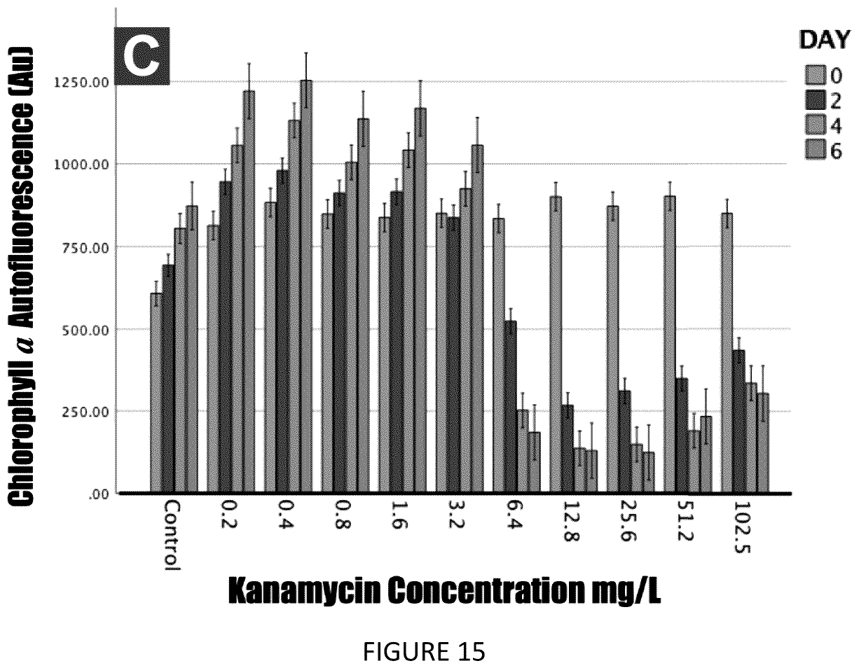

Strain B481-SD treated with ampicillin ranging from 0.2 to 25.6 mg/L exhibited significant increases in phycocyanin and chlorophyll α autofluorescence; however, a significant decrease was detected at 51.2 and 102.5 mg/L (p<0.01) (FIGS. 5, 13). By contrast, B481-WT treated with ampicillin exhibited a significant decrease in pigment autofluorescence from 3.2 to 102.5 mg/L compared to the untreated control. We observed a significant reduction of pigment autofluorescence in B481-SD and B481-WT treated with cefotaxime ranging from 0.8 to 102.5 mg/L and 0.2 to 102.5 mg/L respectively (FIGS. 6, 10, 14, 18). A significant reduction in phycocyanin and chlorophyll α autofluorescence was observed in both strains exposed to kanamycin from 6.4 to 102.5 mg/L when compared to the untreated control. However, a significant increase in B481-SD autofluorescence at lower kanamycin concentrations of 0.2 to 3.2 mg/L when compared to the control was observed (FIGS. 7, 15). B481-SD treated with tetracycline reduced phycocyanin and chlorophyll α autofluorescence at concentrations ranging from 102.5 to 410 mg/L, while it ranged from 25.6 to 410 mg/L for B481-WT. A significant increase in pigment autofluorescence was observed in both strains treated with tetracycline from 0.8 to 12.8 mg/L on day 6 (FIGS. 8, 12, 16, 20).

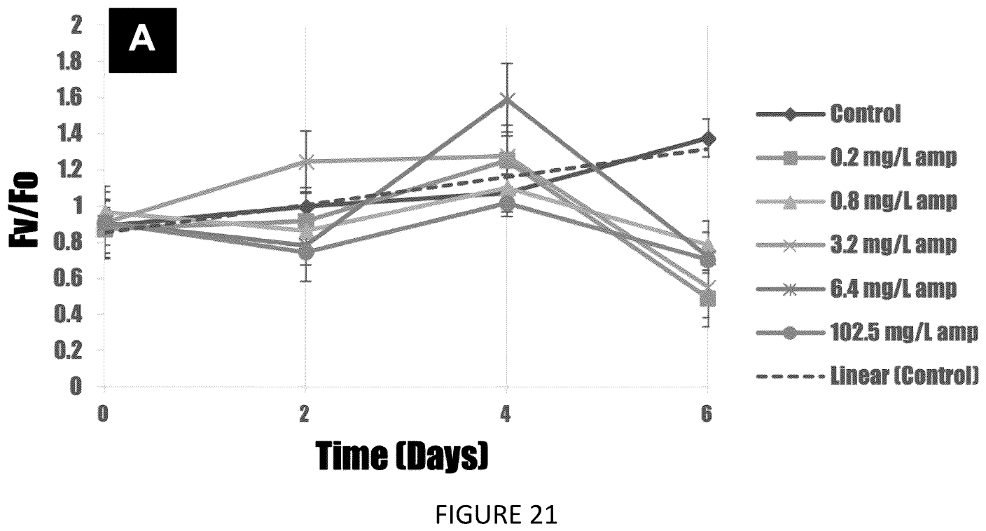

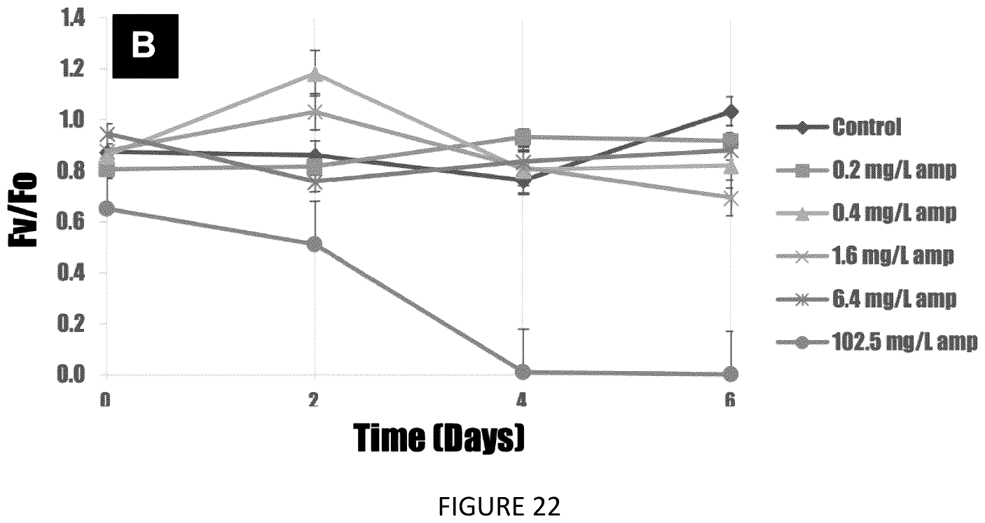

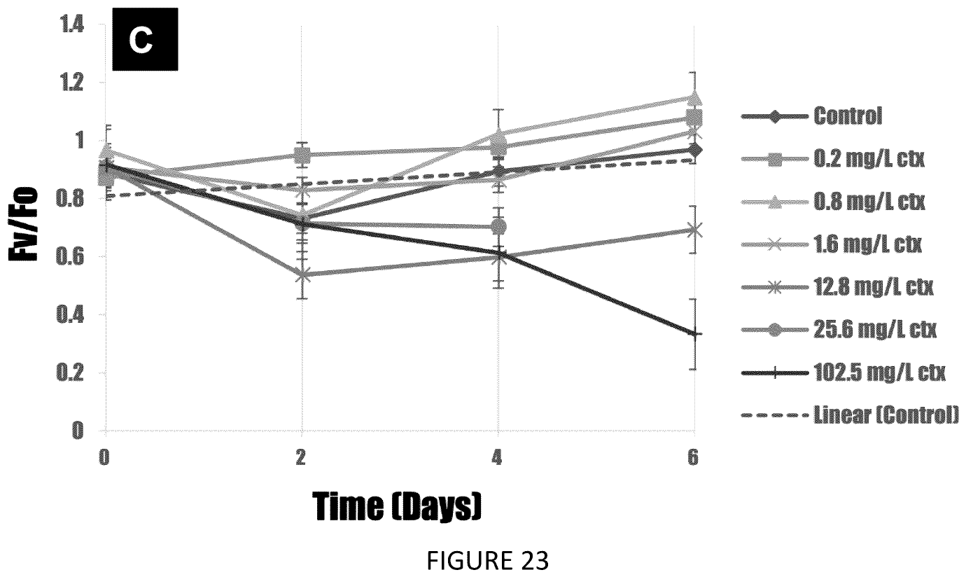

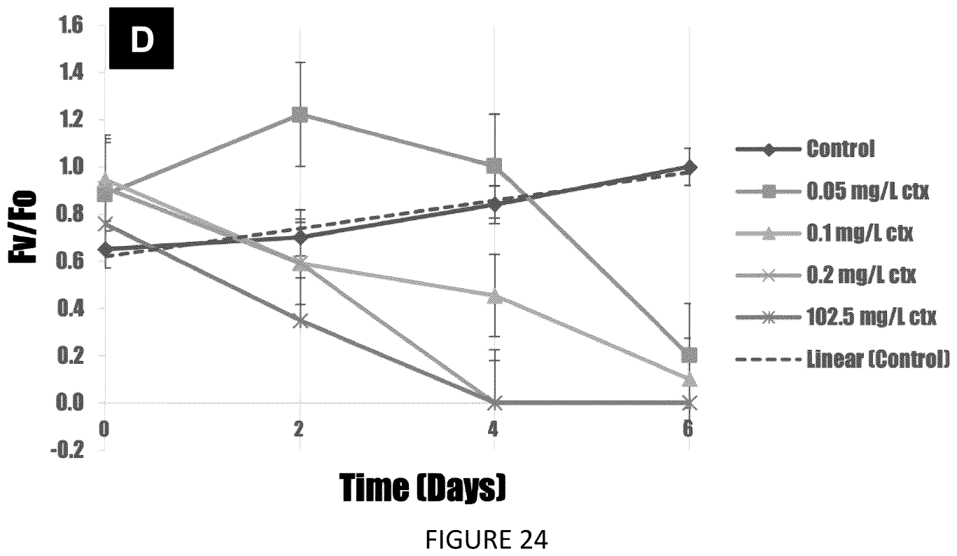

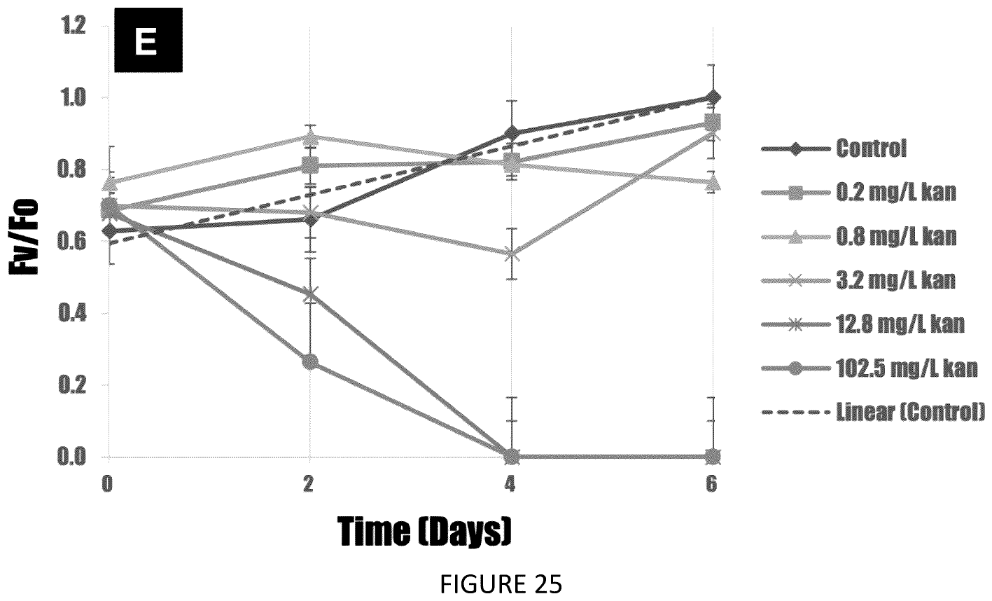

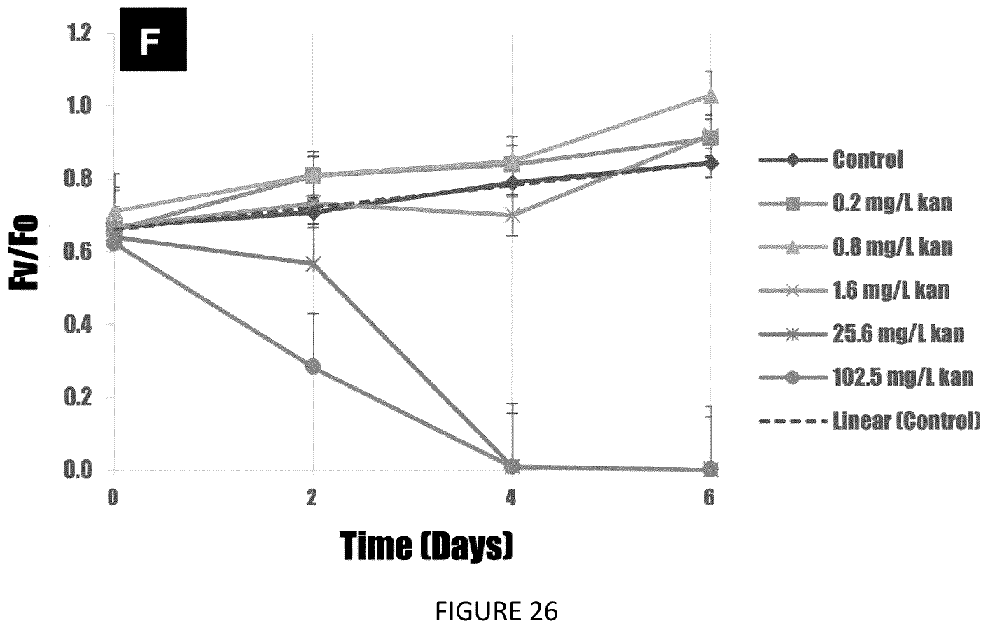

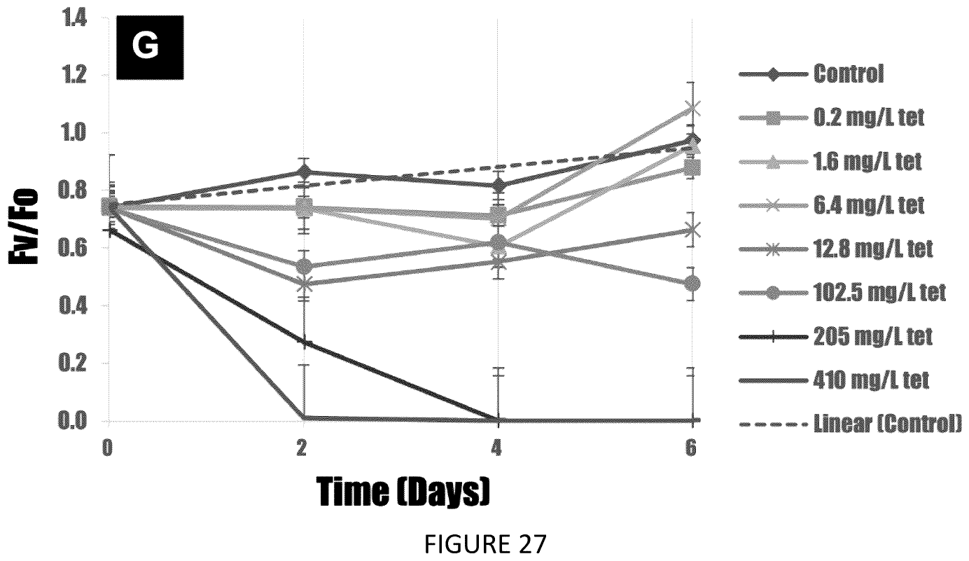

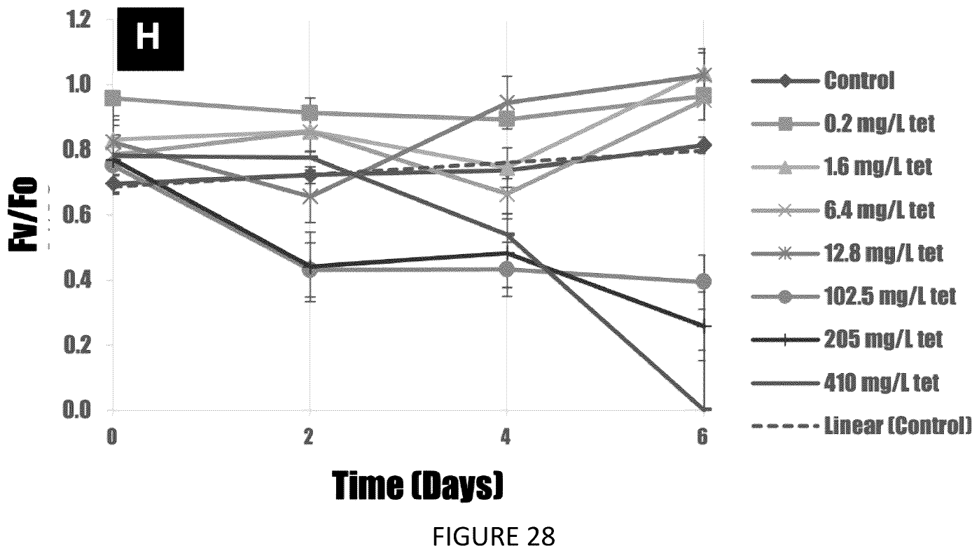

Quantification of photosynthetic efficiency (Fv/Fo) revealed a significant increase in B481-SD strain treated with ampicillin at 0.2 to 3.2 mg/L on day 4 compared to the control (FIG. 21). On the other hand, a significant reduction of Fv/Fo ratios was observed in B-481-SD treated with 51.2 and 102.8 mg/L ampicillin on day 6 (FIGS. 21, 22). A substantial reduction in the Fv/Fo ratios in cefotaxime-treated cells was observed, with no recovery of B481-SD and B481-WT at concentrations higher than 1.6 mg/L and 0.05 mg/L respectively (FIGS. 23, 24). While we observed a significant decrease in Fv/Fo ratio in both strains treated with kanamycin from 1.6 to 102.5 mg/L, a significant increase in B481-WT at concentrations ranging from 0.2 to 1.6 mg/L kanamycin compared with the control group was noted (FIG. 26). We also observed a decrease in Fv/Fo ratios in both strains exposed to tetracycline concentrations higher than 102.5 mg/L (p<0.05) (FIGS. 27, 28).

Assessment of membrane integrity as a measure of extracellular LDH enzyme activity revealed a linear correlation (data not shown). In both strains treated with antibiotics, LDH activity correlated to phycocyanin and chlorophyll α accumulation in a dose-dependent manner, indicating a positive correlation between increased metabolic activity and membrane permeability prior to complete cell destruction. Membrane permeability detected by lactate dehydrogenase assay indicated maximal activity at 0.8 mg/L ampicillin, kanamycin, and tetracycline treatments on day 6.

Total lipids were extracted and GC×GC/TOF-MS analysis of transesterified lipids in F. diplosiphon treated with optimal ampicillin were performed. B481-SD cultures were grown under optimal conditions and harvested on day 15. Total lipids were extracted using the chloroform: methanol method described by Folch et al. (1957). Briefly, cultures were pelleted and lyophilized, and 15 mL methanol/chloroform (2:1, v/v) solvent was added to 100 mg dry weight of cells. The mixture was homogenized, incubated on ice, and centrifuged at 3000 rpm for 10 min to collect the organic phase. The biomass residue was re-extracted thrice with 4 mL of methanol and 2 mL of chloroform. The lower organic phase was dried in a rotary evaporator (Heidolph, IL, USA), and the total lipid content was estimated by gravimetry.

Simultaneous extraction and transesterification of F. diplosiphon lipids were performed according to the method described by Wahlen et al. (2011). Cells grown in 0.8 mg/L ampicillin and the control were lyophilized (100 mg), dissolved in 3 mL methanol containing 1.8% (v/v) sulfuric acid, and exposed to 80° C. for 20 min in a commercial multimode scientific microwave (CEM Corporation, USA) with a maximum power output set at 25W per sample. The reaction was quenched with 4 ml chloroform, washed in distilled water, and centrifuged at 2000 rpm for phase separation. The chloroform phase containing FAMEs and lipids was transferred to a new flask, and the remaining biomass was washed twice with 2 mL chloroform and mixed. The extracted FAMEs were subjected to high-resolution two-dimensional gas chromatography-time of flight mass spectrometry (GC×GC/TOF-MS) as described by Tabatabai et al. (2018).

Statistical Analysis: Repeated ANOVA and Tukey's multiple comparison tests including Pearson's correlation were used to analyze F. diplosiphon sensitivity to different antibiotic treatments at each sampling point. SPSS 28.0 (IBM Corporation, Armonk, United States) was also used to analyze and plot the data.

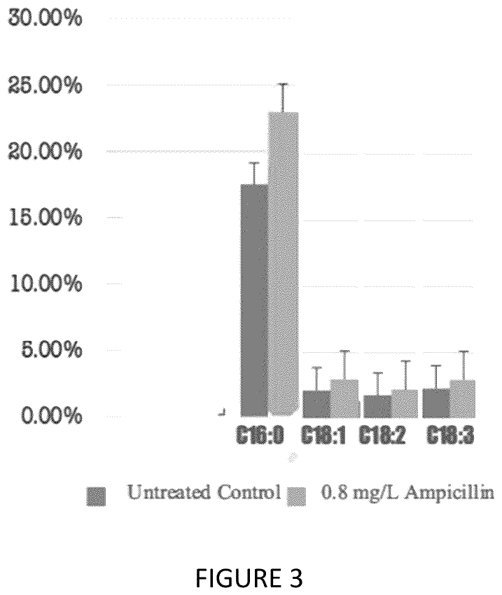

Using conventional methods to quantify lipid subtypes and lipid yield produced, we observed a slight increase in total lipid content of in F. diplosiphon treated with 0.8 mg/L of ampicillin compared to the control; however, it was not statistically different (p>0.05) from the relative abundance of FAMEs quantified by GC×GC/TOF-MS (FIG. 3).

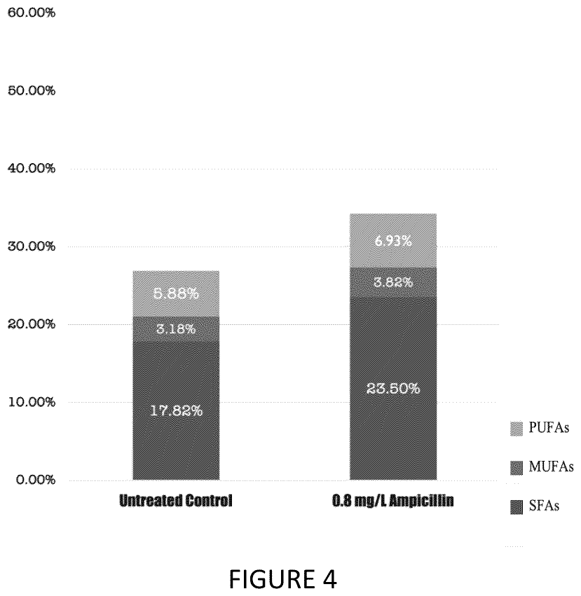

A crucial criterion for biofuel quality is the quantity of fatty acid saturation. To achieve ideal biodiesel properties, the most preferred group of fatty acids are the monounsaturated fatty acids (MUFAs), followed by polyunsaturated fatty acids, and lastly the saturated fatty acids (SFA). We observed an augmentation of SFAs in 0.8 mg/L ampicillin when compared to the control (FIG. 4). SFAs are preferred in extreme conditions due to their resilience.

In the transesterified lipids of F. diplosiphon treated with 0.8 mg/L ampicillin, methyl ester of hexadecanoic acid (C16: 0), methyloctadecenoate (C18: 1) methyl octadecadienoate (C18: 2) and ç-Linolenic acid (C18: 3) levels were significantly higher (p<0.05) than the untreated control. We conclude that low dose antibiotic is effective in increasing FAME compositions.

1. A method for increasing pigmentation and/or bioproduct production in cyanobacteria comprising growing cultures of said cyanobacteria in the presence of non-lethal levels of antibiotics.

2. The method of claim 1, wherein said cyanobacteria is F. diplosiphon.

3. The method according to claim 1, wherein said cyanobacteria is F. diplosiphon strain B481-SD or B481-WT.

4. The method according to claim 1, wherein said antibiotic is a β-lactam, an aminoglycoside or tetracycline.

5. The method according to claim 1, wherein said antibiotic is ampicillin, tetracycline, or kanamycin.

6. The method according to claim 1, wherein optimal antibiotic concentration is determined by lactate dehydrogenase activity.

7. The method according to claim 1, wherein said bioproduct production is lipid production and includes production of one or more of saturated fatty acids, polyunsaturated fatty acids, and monounsaturated fatty acids.

8. The method according to claim 1 wherein phycocyanin and chlorophyll α autofluorescence is significantly increased at concentrations from 0.2 to 25.6 mg/L ampicillin in B481-SD as compared to control cultures of B481-SD grown in the absence of antibiotic.

9. The method according to claim 1 wherein a significant increase in B481-SD autofluorescence occurs at kanamycin concentrations of 0.2-3.2 mg/L as compared to control cultures of B481-SD grown in the absence of kanamycin.

10. The method according to claim 1 wherein a significant increase in pigment autofluorescence in F. diplosiphon strains B481-SD and B481-WT treated with tetracycline from 0.8 to 12.8 mg/L on day 6.

11. The method according to claim 1 wherein LDH activity correlates to phycocyanin and chlorophyll α accumulation in a dose-dependent manner in B481-SD and B481-WT.

12. A composition comprising cyanobacteria and a non-lethal amount of an antibiotic.

13. The composition of claim 12, wherein said cyanobacteria is F. diplosiphon.

14. The composition of claim 12, wherein said cyanobacteria is F. diplosiphon strain B481-SD or B481-WT.

15. The composition of claim 12, wherein said antibiotic is a β-lactam, an aminoglycoside or tetracycline.

16. (canceled)

17. The composition of claim 12, wherein said antibiotic is present in concentrations of about 0.2 mg/L to about 0.8 mg/L ampicillin, tetracycline, and kanamycin.

18. (canceled)

19. (canceled)

20. The composition of claim 12, further comprising increased amounts of bio-products as compared to cyanobacteria compositions grown in the absence of antibiotic.

21. The composition of claim 12, further comprising fatty acid methyl ester in increased amounts as compared to cyanobacteria compositions grown in the absence of antibiotic.

22. The composition of claim 21, wherein said fatty acid method esters comprising saturated fatty acids, polyunsaturated fatty acids, and monounsaturated fatty acids.

23. (canceled)

24. (canceled)

25. (canceled)

26. The composition of claim 21, comprising increased amounts of hexadecanoic acid (C16:0), methyloctadecenoate (C18:1) methyl octadecadienoate (C18:2) and çLinolenic acid (C18:3) as compared to cyanobacteria compositions grown in the absence of antibiotic.
